(19) United States
(12) Patent Application Publication
(10) Pub. No.: US 2024/0382422 A1
(43) Pub. Date: Nov. 21, 2024

(54) POLYMERIC MICELLE NANOPARTICLES FOR ON-DEMAND CELLULAR PACKAGING OF EXTRACELLULAR VESICLES

(71) Applicant: University of Florida Research Foundation, Incorporated, Gainesville, FL (US)

(72) Inventor: Mei He, Gainesville, FL (US)

(21) Appl. No.: 18/696,420

(22) PCT Filed: Oct. 25, 2022
(86) PCT No.: PCT/US2022/078633

§ 371 Date: Mar. 28, 2024

Related U.S. Application Data
(60) Provisional application No. 63/272,786, filed on Oct. 28, 2021.

(51) Int. Cl. A61K 9/107; A61K 9/50; A61K 45/06; A61K 47/69; C12N 5/0784

(52) U.S. Cl. CPC A61K 9/1075; A61K 9/5068; A61K 45/06; A61K 47/6907; C12N 5/0639; C12N 2501/998

(57) ABSTRACT

Described are polymeric micelle nanoparticles and their use in producing extracellular vesicles containing a cargo molecule. The cargo-loaded extracellular vesicles can be used as therapeutic delivery vectors.

### CROSS-REFERENCE TO RELATED APPLICATIONS

This application is a National Stage Application, filed under 35 U.S.C. § 371, of International Application No. PCT/US2022/078633, filed Oct. 25, 2022, which claims the benefit of U.S. Provisional Application No. 63/272,786, filed Oct. 28, 2021, each of which is incorporated herein by reference.

### STATEMENT REGARDING FEDERALLY SPONSORED RESEARCH OR DEVELOPMENT

This invention was made with government support under grant number R35 GM133794 awarded by the National Institutes of Health. The government has certain rights in the invention.

### BACKGROUND

Developing therapeutic cancer vaccines to modulate immune responses for cancer treatment has gained increasing interest. However, to date, such cancer vaccines suffer from weak immunogenicity, low specificity, and off-target effects. Developing novel cancer vaccines is needed for precise control of anti-tumor immune responses.

Exosomes are small extracellular vesicles (30-150 nm) that originate from the endocytic compartment of the cell. These exosomes can serve similar biological functions to their parent cells and have emerged and a possible therapeutic delivery and vaccine platform. Low exosome secretion from parent cells, along with limited packaging capacity remain significant obstacle in meeting clinical utility.

The ability to form extracellular vesicles having a desired cargo molecule (designer extracellular vesicles) would further increase the utility of extracellular vesicles, including and exosomes.

### SUMMARY

Described are polymeric micelle nanoparticles useful for forming extracellular vesicles transfection tools that can be used to enhance the site-specific packaging of extracellular vesicles with a cargo molecule of choice.

Described are polymeric micelle nanoparticles comprising a first amphiphilic block copolymer having at least one reversible linkage to at least one cargo molecule and a second amphiphilic block copolymer covalently linked to a targeting molecule. The first amphiphilic block copolymer and the second amphiphilic block copolymer can be diblock or triblock copolymers. The first amphiphilic block copolymer and the second amphiphilic block copolymer each comprise a hydrophobic block and a hydrophilic block. In some embodiments, the polymeric micelle nanoparticles are spherical in aqueous solution. In some embodiments, the polymeric micelle nanoparticle are stable in an aqueous solution at about pH 7.4.

The hydrophobic blocks of the first and second amphiphilic block copolymers can be the same or they can be different. The hydrophobic blocks of the first and second amphiphilic block copolymers can comprise block polymers of the same monomer or different monomers. The hydrophilic blocks of the first and second amphiphilic block copolymers can be the same or they can be different. The hydrophilic blocks of the first and second amphiphilic block copolymers comprises block polymers of the same monomer or different monomers.

In some embodiments, one or more cargo molecules are linked to the hydrophilic block of the first amphiphilic block copolymer via the one or more reversible linkages. The reversible linkage can be cleaved in response to a drop in pH, an exposure to light, a reducing agent, a change in temperature, an enzyme, or an added agent. The cargo molecule can be, but is not limited to, a pharmaceutical molecule, a therapeutic compound, a drug, a small molecule, a hormone, a cytokine, polypeptide, antibody, an antibody fragment, an antigen-binding polypeptide or molecule (e.g., scFv), or a nucleic acid molecule, or derivative thereof.

In some embodiments, the targeting molecule is linked to the hydrophilic block of the second amphiphilic block copolymer. The targeting molecule can be, but is not limited to, a compound having affinity for a cell surface molecule, a cell receptor ligand, an antibody, an antibody fragment, or an antigen-binding polypeptide. A cell receptor ligand can be, but is not limited to, a carbohydrate, a glycan, a saccharide, a galactose, a galactose derivative, mannose, a mannose derivative, a vitamin, a folate, a biotin, an aptamer, a peptide, an RGD-containing peptide, an insulin, an epidermal growth factor, or a transferrin. Targeting molecules, in combination with the cargo molecule can be used in precision medicine or personalized medicine.

In some embodiments, the first amphiphilic block copolymer comprises PCLₙ₁-PEGₘ₁ wherein n1 is an integer from 2 to 250 and m1 is an integer from 2 to 750 and the second amphiphilic block copolymer comprises PCLₙ₂-PEGₘ₂ wherein n1 is an integer from 2 to 250 and m1 is an integer from 2 to 750. In some embodiments, n1 is about 26, m1 is about 66, n2 is about 26, and m2 is about 111.

Also described are methods of loading an extracellular vesicle with a cargo molecule comprising: contacting a cell with the described polymeric micelle nanoparticles; incubating the polymeric micelle nanoparticles with the cell for a period of time sufficient to enable endocytosis of the polymeric micelle; and exposing the cell to conditions suitable for cleavage of the reversible linkage.

Described are methods of using polymeric micelle nanoparticles to produce extracellular vesicles having a cargo molecule of choice. The loaded extracellular vesicles can then be used in therapies, including delivery of therapeutic molecules to a subject.

Described are methods of forming extracellular vesicles containing a cargo molecule comprising: contacting a cell with the described polymeric micelle nanoparticles; incubating the polymeric micelle nanoparticles with the cell for a period of time sufficient to enable endocytosis of the polymeric micelle; exposing the cell to conditions suitable for cleavage of the reversible linkage; and collecting extracellular vesicles from the cell. The cell can be incubated with the polymeric micelle nanoparticles for about 10 minutes to about 2 hours. The cell can be, but is not limited to, eukaryotic cell, a mammalian cell, a stem cell, an adult stem cell, an embryonic stem cell, a neural stem cell, an immune cell, a mesenchymal stem/stromal cell, an antigen-presenting cell, a dendritic cell (DC), an immature DC, a mature DC, a bone marrow derive DC, a macrophage, a B lymphocyte (B cell), a T cell, an endothelial cell, or a fibroblast. The cargo-loaded extracellular vesicles can then be used for research or therapeutic purposes.

In some embodiments, the reversible linkage linking the cargo molecule to the first amphiphilic block copolymer is a near UV sensitive linkage and exposing the cell to near UV light results of cleavage of the cargo molecule from the first amphiphilic block copolymer. Release of the cargo molecule allows the cell to package the cargo molecule into extracellular vesicles.

### BRIEF DESCRIPTION OF THE DRAWINGS

FIG. 1. Illustration of using polymer micelle nanoparticles having a UV-sensitive linkage to a cargo molecule to generate designer exosomes.

FIG. 2. Exemplary amphiphilic block copolymers for forming polymeric micelle nanoparticles.

FIG. 3A. Characterization of PPPG-PPM polymeric micelle nanoparticles. ¹H-NMR spectrum of gp100-PC in DMSO-D6 (PC=photo-cleavable linkage).

FIG. 3B. Characterization of PPPG-PPM polymeric micelle nanoparticles. ¹H-NMR spectrum of PPPG in DMSO-D6.

FIG. 3C-E. Characterization of PPPG-PPM polymeric micelle nanoparticles. (C) Number size distribution of PPPG-PPM polymeric micelle nanoparticles as determined by dynamic light scattering. (D) Zeta potential distribution of PPPG-PPM polymeric micelle nanoparticles as determined by dynamic light scattering. (E) Transmission electron micrograph of PPPG-PPM polymeric micelle nanoparticles after staining with 2% phosphotungstic acid.

FIG. 4. Diagram illustrating synthesis of PPPG.

FIG. 5. Diagram illustrating synthesis of PPG

FIG. 6A. ¹H-NMR spectrum of the gp100 peptide in DMSO-D6.

FIG. 6B. ¹H-NMR spectrum of PCL-PEG-N3 in DMSO-D6.

FIG. 7A. ¹H-NMR spectrum of PCL-PEG-NHS in DMSO-D6.

FIG. 7B. ¹H-NMR spectrum of PPG in DMSO-D6.

FIG. 8. FTIR spectra of gp100, PC linker, and gp100-PC-linker.

FIG. 9. FTIR spectra of gp100-PC-linker, PCL-PEG-N3, and PCL-PEG-PC-gp100.

FIG. 10. FTIR spectra of gp100-PC-linker, PCL-PEG-NHS, and PCL-PEG-gp100.

FIG. 11A-B. Size distribution (A) and Zeta potential (B) of polymeric micelle nanoparticles.

FIG. 11C. Transmission Electron Micrographs of control polymeric micelles nanoparticles.

FIG. 12. Immunofluorescence staining of mature dendritic cells (mDCs) with Cy3-labeled anti-EEA antibody post-incubation with Cy5.5-labeled polymeric micelle nanoparticles at different time points.

FIG. 13. Immunofluorescence staining of mDCs with Cy3-labeled anti-Rab7 antibody. mDCs were pretreated with NPs for different time points.

FIG. 14. Immunofluorescence staining of mDCs with Cy3-labeled anti-Lamp1 antibody. mDCs were pretreated with NPs for different time points.

FIG. 15. Graph illustrating dendritic cell viability following incubation with polymeric micelle nanoparticles and exposure to light to cleave the photosensitive linkers in the polymeric micelle nanoparticles.

FIG. 16. Images showing colocalization of polymeric micelle nanoparticles in early endosomes, later endosomes, and lysosomes at various times Scale bar is 5 mm.

FIG. 17. Graph illustrating uptake efficiency uptake of peptide alone (first bar in each series), untargeted polymeric micelle nanoparticles (nanoparticle-COOH, second bar in each series), and targeted polymeric micelle nanoparticles (nanoparticles-MAN, third bar in each series).

FIG. 18A. Graphs illustrating size of EVs harvested from untreated dendritic cells or dendritic cells treated with gp100, or dendritic cells loaded polymeric micelle nanoparticles (ExoPack) and exposure to light for 0 minutes, 1 min, or 30 min.

FIG. 18B. Graphs illustrating zeta potential of EVs harvested from untreated dendritic cells or dendritic cells treated with gp100, or dendritic cells loaded polymeric micelle nanoparticles (ExoPack) and exposure to light for 0 minutes, 1 min, or 30 min.

FIG. 18C. Graphs illustrating particle concentration of EVs harvested from untreated dendritic cells or dendritic cells treated with gp100, or dendritic cells loaded polymeric micelle nanoparticles (ExoPack) and exposure to light for 0 minutes, 1 min, or 30 min.

FIG. 19. Graphs illustrating (A) Cellular uptake of loaded polymeric micelle nanoparticles (ExoPack) in dendritic cells increases over time; and (B) ExoPack is highly biocompatible and retains significant cell viability over time

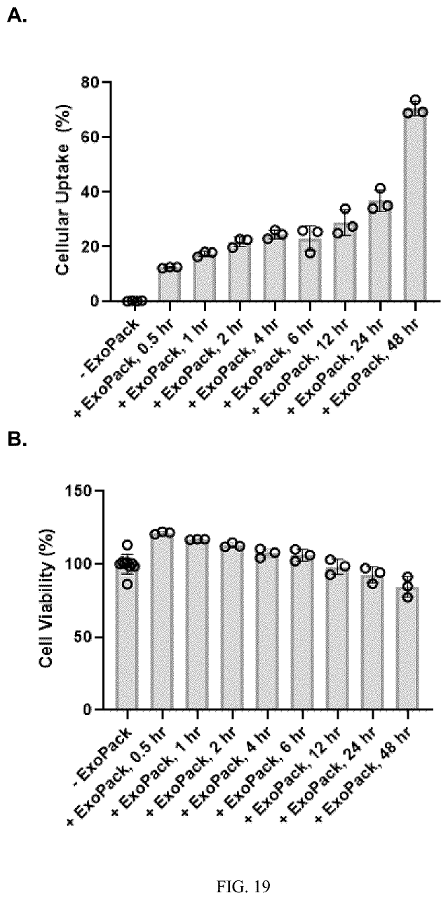

FIG. 20. Transmission electron microscopy images showing the morphology of native EVS and EVs harvested from dendritic cells following nanotransfection with polymeric micelle nanoparticles and exposure to light for 1 or 30 minutes to release cargo.

FIG. 21. Graph illustrating concentration of gp100 in EVs harvested from untreated dendritic cells, dendritic cells treated with gp100 alone, or dendritic cells nanotransfected with polymeric micelle nanoparticles containing gp100 and either exposed or not exposed to light to release gp100 from the nanoparticles.

### DETAILED DESCRIPTION

### I. Definitions

Before describing the present teachings in detail, it is to be understood that the disclosure is not limited to specific compositions or process steps, as such may vary. As used in this specification and the appended claims, the singular form “a,” “an,” and “the” include plural references unless the context clearly dictates otherwise. Thus, for example, reference to “a peptide” includes a plurality of peptides and the like. The conjunction “or” is to be interpreted in the inclusive sense, i.e., as equivalent to “and/or,” unless the inclusive sense would be unreasonable in the context.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

“Subject” refers to an animal, such as a mammal, for example a human. The methods described herein can be useful in both humans and non-human animals. In some embodiments, the subject is a mammal (such as an animal model of disease), and in some embodiments, the subject is human.

An “Antigen-Presenting Cell” (APC) is a cell that displays antigen complexed with major histocompatibility complex II (MHCII) on their surfaces. APCs can process external antigens and present them to other immune cells, such as T cells. Macrophages, B cells and dendritic cells (professional antigen presenting cells) are naturally occurring professional APCs. An APC may also express one or more co-stimulatory molecules.

“Dendritic cells” are antigen-presenting cells having the broadest range of antigen presentation and the ability to activate naïve T cells. Their main function is to process antigen material and present it on the cell surface to T cells. DCs present antigen to both helper and cytotoxic T cells.

“Immune therapy” or “Immunotherapy” is the treatment of disease, such as cancer, by activating or suppressing the immune system. Immunotherapies can be designed to elicit or amplify an immune response.

### II. Polymeric Micelle Nanoparticles

Described are functionalized polymeric micelle nanoparticle capable of delivering a cargo molecule to a cell to be incorporated into an extracellular vesicle. The described polymeric micelle nanoparticles comprise a first block copolymer having at least one reversible linkage to at least one cargo molecule and a second block copolymer linked to at least one targeting molecule, wherein the first and second block copolymers each comprise a hydrophobic block and a hydrophilic block (i.e., the block copolymers are amphiphilic block copolymers). The hydrophobic block of the first block polymer can associate with the hydrophobic block of the second block polymer in forming a micelle. The first and second block copolymers can be the same or they can be different. The hydrophobic blocks of the first and second block copolymers can be the same or they can be different. The hydrophobic blocks of the first and second block copolymers can be synthesized from the same monomer or from different monomers. If the hydrophobic blocks of the first and second block copolymers are be synthesized from the same monomers, the hydrophobic blocks of the first and second block copolymers can be the same length or different lengths. The hydrophilic blocks of the first and second block copolymers can be the same or they can be different. The hydrophilic blocks of the first and second block copolymers can be synthesized from the same monomer or from different monomers. If the hydrophilic blocks of the first and second block copolymers are be synthesized from the same monomers, the hydrophilic blocks of the first and second block copolymers can be the same length or different lengths. In some embodiments, the first and second block copolymers are diblock amphiphilic block copolymers. In some embodiments, the first and second block copolymers have a hydrophilic volume fraction, f, greater than 45%.

In some embodiments, polymeric micelle nanoparticles are described comprising polymers represented by AₐB_{b}-(L-D)ₓ and A′_{a′}B′_{b′}-T_{y} wherein AₐB_{b} and A′_{a′}B′_{b′} are amphiphilic block copolymers (e.g., polyA-block-polyB and polyA′-block-polyB′), Aₐ and A′_{a′} are hydrophobic polymers, B_{b} and B′_{b′ are hydrophilic polymers, A and A′ are monomers which when polymerized form a hydrophobic polymer, B and B′ are monomers which when polymerized form a hydrophilic polymer, a and a′ are integers from} 2 to about 250, b and b′ are integers from 2 to about 750, x and y are integers greater than or equal to 1, L comprises a reversible linkage, D comprises a cargo molecule, and T comprises a targeting molecule. AₐB_{b} and A′_{a′}B′_{b′ can be the same or different. A}ₐ and A′_{a′} can be the same or different. B_{b} and B′_{b′ can be the same or different. A and A′ can be the same or different. B and B′ can be the same or different. a and a′ can be the same or different. b and b′ can be the same or different. In some embodiments, the length of B}_{b} and B′_{b′ exceed the length of A}ₐ and A_{a′} respectively.

The hydrophobic blocks of the first and second block copolymers can be formed from monomers that are polar or non-polar, charged or uncharged, provided the block is overall hydrophobic. Exemplary hydrophobic monomers include, but are not limited to, polycaprolactone (PCL), polyvalerolactone (PVL), poly(lactide-co-glycolide) (PLGA), polylactic acid (PLA), poly(L-lactide), polybutyrolactone (PBL), polyglycolide, polypropiolactone (PPL), polyacrylate, polybutylene oxide, a polybutadiene, poly(allyl glycidyl ether), poly(aspartate ester), and polyaminoacid (e.g., F poly(L-lysine)). A hydrophobic block can be a homopolymer or a heteropolymer.

The hydrophilic blocks of the first and second block copolymers comprise a plurality of hydrophilic monomers. The hydrophilic monomers can be charged or uncharged. Charged hydrophilic monomers can be anionic, cationic, or zwitterionic. In some embodiments, the hydrophilic monomers are uncharged. Exemplary hydrophilic blocks include, but are not limited to, polypropylene glycol (PEG), polyethylene oxide (PEO), poloxamer, polyaminoacid, poly-7-glutamine acid (γ-PGA), polyacrylamides, polyvinylpyrrolidone, hyaluronic acid (HA), zwitterionic polymer, and polysaccharide. A hydrophobic block can be a homopolymer or a heteropolymer.

The monomers and length of the hydrophobic and hydrophilic blocks are selected such that the first and second block copolymers auto assemble in aqueous solution to form spherical polymeric micelles. Polymeric micelles are composed of aggregates of amphiphilic co-polymers (consisting of both hydrophilic and hydrophobic monomer units) assembled into hydrophobic cores, surrounded by a corona of hydrophilic polymeric chains exposed to the aqueous environment. In some embodiments, the length of the hydrophilic block is longer than the length of the hydrophobic block. The spherical polymeric micelle can be about 10 to about 200 nm in diameter, about 10 to about 100 nm in diameter, about 10 to about 80 nm in diameter, about 10 to about 70 nm in diameter, about 10 to about 60 nm in diameter, or about 10 to about 50 nm in diameter. In some embodiments, the spherical polymeric micelle can be 20 nm±15 nm, 20 nm±10 nm, 20 nm±5 nm, or about 20 nm in diameter. In some embodiments, the polymeric micelle are stable in aqueous solution at about pH 7.4. In some embodiments, the polymeric micelle are essentially kinetically frozen in aqueous solution.

In some embodiments, the amphiphilic clock copolymers are biodegradable. A polymer may have one or more cleavable bonds. If the cleavable bonds are naturally cleaved under physiological conditions or cellular physiological conditions, the polymer is biodegradable. The biodegradable bond may either be in the main-chain or in a side chain. If the cleavable bond occurs in the main chain, cleavage of the bond results in a decrease in polymer length and the formation of two molecules. If the cleavable bond occurs in the side chain, then cleavage of the bond results in loss of side chain atoms from the polymer. A biodegradable polymer may degrade over time by the action of enzymes, by hydrolytic action and/or by other similar mechanisms in the body. Biodegradable bonds are those bonds which are cleaved by biological processes and include, but are not limited to: esters, phosphodiesters, certain peptide bonds and combinations thereof. Esters undergo hydrolysis and are also catalytically cleaved by esterases. Phosphodiesters are cleaved by nucleases. Peptide bonds are cleaved by peptidases. Biodegradable bonds in the biodegradable polymers may be cleaved, under physiological conditions with a half life of less than 45 min, more than 45 minutes, more than 2 hours, more than 8 hours, more than 24 hours, or more than 48 hours.

The size and shape of the polymeric micelles can be determined by methods known in the art, included, but not limited to, light scattering, scanning electron microscopy (SEM), transmission electron microscopy (TEM), atomic force microscopy (AFM), asymmetric flow field flow fractionation, and small angle neutron scattering.

In some embodiments, the polymeric micelle nanoparticles contain one or more additional amphiphilic block copolymers. The one or more additional amphiphilic block copolymers can be linked to additional cargo molecules via reversible linkages.

A reversible linkage is a connection between two atoms that link one chemical group or segment of interest (e.g., block copolymer) to another chemical group or segment of interest (e.g., cargo molecule) via a covalent bonds. A reversible or labile linkage contains a reversible or labile bond. A reversible linkage may optionally include a spacer that increases the distance between the two joined atoms. A spacer may further add flexibility and/or length to the linkage. A reversible or labile bond is a covalent bond other than a covalent bond to a hydrogen atom that is capable of being selectively broken or cleaved under conditions that will not break or cleave other covalent bonds in the same molecule. More specifically, a reversible or labile bond is a covalent bond that is less stable (thermodynamically) or more rapidly broken (kinetically) under appropriate conditions than other non-labile covalent bonds in the same molecule. Cleavage of a labile bond within a molecule may result in the formation of two or more molecules. Appropriate conditions are determined by the type of labile bond and are well known in organic chemistry. A labile bond can be sensitive to pH, oxidative or reductive conditions or agents, temperature, light, the presence of an enzyme, or the presence of an added agent.

Reversible linkages undergo a chemical transformation (e.g., cleavage) when present in certain physiological conditions or in response to an external stimulus. A physiological condition can be, but is not limited to, decreased pH, such as in an endosome. An external stimulus can be, but is not limited to, an added a compound, light (including UV or near UV light), or heat. It is known in the art, that the conditions under which a reversible linkage will undergo transformation can be controlled by altering the chemical constituents of the molecule containing the reversible linkage. For example, addition of particular chemical moieties (e.g., electron acceptors or donors) near the labile group can affect the particular conditions (e.g., pH) under which chemical transformation will occur. A pH-labile bonds is a labile bond that is selectively broken under acidic conditions (pH<7), such as occurs in cellular endosomes and lysosomes. A photosensitive bond is a bond that is selectively broken when exposed to light, such as UV or near UV light. A thermosensitive bond is a labile bone that is broken when exposed to heat. Photocleavable linkers include, but are not limited to, arylcarbonylmethyl groups, aromatic ketones, o-alkylphenacyl groups, p-hydroxyphenacyl groups, benzoin groups, nitroaryl groups, o-nitrobenzyl groups, o-nitro-2-phenethyloxycarbonyl groups, o-nitroanilides, coumarin-4-ylmethyl groups, arylmethyl groups, o-hydroxyarylmethyl groups, metal-containing photoremovable groups, pivaloyl group, esters of carboxylic acids, arylsulfonyl groups, carbanion-mediated groups, sisyl groups, 2-hydroxycinnamyl groups, α-keto amides, α,β-unsaturated anilides, methyl(phenyl)thiocarbamic acid, 2-pyrrolidino-1,4-benzoquinone group, triazine and arylmethyleneimino groups, and xanthene and pyronin groups (Klan et al. “Photoremovable Protecting Groups in Chemistry and Biology:Reaction Mechanisms and Efficacy” Chemical Reviews 2013, 113:119-191; and Hansen et al. “Wavelength-selective cleavage of photoprotecting groups: strategies and application in dynamic systems” Chem Soc Rev. 2015 44:3358-3377). In some embodiments, the photocleavable linker can comprises a nitrobenzyl group.

Exemplary reversible linkages include, but are not limited to, a linkage comprising a photocleavable group, a disulfide bond, an acetal, a ketal, an enol ether, an enol esters, an 2,3-disubstituted maleamic acid (maleamates), an imine, an iminium, an enamine, a hydrozone, a silazene, a silane, silyl ether, a silyl enol ether, and a siloxane.

In some embodiments, the reversible linkage is photosensitive. The photosensitive reversible linkage can be, but is not limited to, a linkage that is sensitive to light having a wavelength of 300-700 nm. The photosensitive reversible linkage can be, but is not limited to, a linkage that is sensitive to near UV light. Near UV light comprises light having a wavelength of about 315 nm to about 500 nm, of about 340 nm to about 390 nm, of about 365 nm, about 405 nm, or about 488 nm. In some embodiments, the near UV light has a wavelength of about 365 nm.

A cargo molecule can be any molecule that is desired to be incorporated into an extracellular vesicle. A cargo molecule can be, but is not limited to, a pharmaceutical molecule, a therapeutic compound, a drug, a proteolysis targeting chimeric (PROTAC), a small molecule, a hormone, a cytokine, polypeptide, antibody, an antibody fragment, an antigen-binding polypeptide or molecule (e.g., scFv), an immunotherapeutic, a cancer immunotherapeutic, or a nucleic acid molecule or derivative thereof. Nucleic acid molecules or derivatives thereof include, but are not limited to antisense oligonucleotides, siRNAs, and miRNAs. Polypeptides include, but are not limited to, proteins, antigens, tumor antigens, pathogenic antigens, viral antigens, bacterial antigens, fungal antigens, parasitic antigens, cytokines, and immune co-stimulators. A cargo molecule can also be a targeting ligand that can direct an extracellular vesicle to a target cell in vivo.

A targeting molecule enhances association of a polymeric micelle with a target cell. In some embodiments, a targeting molecule has affinity for target cell. A targeting molecule can also enhance internalization of a polymeric micelle. In some embodiments, the targeting molecule facilitates or enhances receptor mediated endocytosis of the polymeric micelle. A targeting molecule may be monovalent, divalent, trivalent, tetravalent, or have higher valency. Targeting molecules include, but are not limited to, compounds with affinity to cell surface molecule, cell receptor ligands, antibodies, antibody fragments, and antibody mimics with affinity to cell surface molecules. Cell receptor ligands include, but are not limited to, carbohydrates, glycans, saccharides (including, but not limited to: galactose, galactose derivatives, mannose, and mannose derivatives), vitamins, folate, biotin, aptamers, and peptides (including, but not limited to: RGD-containing peptides, insulin, epidermal growth factor, and transferrin). In some embodiments, the targeting molecule is a ligand for a receptor on a target cell. In some embodiments, a targeting molecule enhances or facilitates cargo loading into exosomes at the endosomes via the endosomal-sorting complex required for transport (ESCRT) machinery.

In some embodiments, the cellular receptor ligand comprises p-aminophenyl-α-D-mannopyranoside (MAN). The MAN modification on the surface of polymeric micelles enhances cellular internalization via mannose receptor-mediated endocytosis uptake by dendritic cells.

In some embodiments, the polymeric micelle nanoparticles comprise: PCLₙ₁-b-PEGₘ₁-PC-gp100 (PPPG) and PCLₙ₂-b-PEGₘ₂-p-aminophenyl-α-D-mannopyranoside (PPM), wherein n1 is an integer from 2 to 250, m1 is an integer from 2 to 750, n2 is an integer from 2 to 250, m2 is an integer from 2 to 750, and wherein PC is a photocleavable linkage. In some embodiments, m1 is less than m2.

In some embodiments, the polymeric micelle nanoparticles comprise: PCL₂₆-b-PEG₆₆-PC-gp100 (PPPG) and PCL₂₆-b-PEG₁₁₁-p-aminophenyl-α-D-mannopyranoside (PPM).

In some embodiments, the polymeric micelle nanoparticles comprise: PCLₙ₁-b-PEGₘ₁-PC-Cargo molecule and PCLₙ₂-b-PEGₘ₂-p-aminophenyl-α-D-mannopyranoside (PPM), wherein n1 is an integer from 2 to 250, m1 is an integer from 2 to 750, n2 is an integer from 2 to 250, m2 is an integer from 2 to 750. In some embodiments, m1 is less than m2.

In some embodiments, the polymeric micelle nanoparticles comprise: PCL₂₆-b-PEG₆₆-PC-Cargo molecule and PCL₂₆-b-PEG₁₁₁-p-aminophenyl-α-D-mannopyranoside (PPM).

In some embodiments, the polymeric micelle nanoparticles comprise: PCLₙ₁-b-PEGₘ₁-PC-Cargo molecule and PCLₙ₂-b-PEGₘ₂-Targeting molecule, wherein n1 is an integer from 2 to 250, m1 is an integer from 2 to 750, n2 is an integer from 2 to 250, m2 is an integer from 2 to 750. In some embodiments, m1 is less than m2.

In some embodiments, the polymeric micelle nanoparticles comprise: PCL₂₆-b-PEG₆₆-PC-Cargo molecule, PCL₂₆-b-PEG₁₁₁-Targeting molecule.

The described polymeric micelle nanoparticles are small in hydrodynamic size, with uniform spherical morphology and biocompatibility for rapid cellular uptake and internationalization. The polymeric micelle nanoparticles provide for high-efficiency transfection of cells and loading of a cargo molecule into extracellular vesicles, including exosomes.

### III. Polymeric Micelle Nanoparticle Formulation

Polymeric micelle nanoparticles comprising the described first and second amphiphilic block copolymers can be formed using methods known in the art for forming polymeric micelles.

In some embodiments, the polymeric micelle nanoparticles are formed by direct dissolution by mixing the first and second block copolymers in aqueous solution.

In some embodiments, the first and second amphiphilic block copolymers are dissolved in a non-aqueous solution and added to an aqueous solution. The first and second amphiphilic block copolymers dissolved in the non-aqueous solution can be gradually added to aqueous solution using any method known in the art for performing addition of a non-aqueous solution into an aqueous solution. In some embodiments, the non-aqueous solution containing the first and second amphiphilic block copolymers is added dropwise into the aqueous solution. Alternatively, the first and second amphiphilic block copolymers dissolved in the non-aqueous solution can be rapidly added to aqueous solution. The non-aqueous solution can be, but is not limited to a non-aqueous polar solvent, non-aqueous polar aprotic solvent, dimethyl sulfoxide (DMSO), dihydrolevoglucosenone, dimethylformamide (DMF), gamma-butyrolactone (GBL), N-methyl-2-pyrrolidone (NMP), dimethylacetamide (DMAc), acetonitrile, dichloromethane, or ethyl acetate. The aqueous solution can be, but is not limited to, water, buffer, or cell culture media. The aqueous solution may contain one or more agents that stabilize the polymeric micelle nanoparticles. In some embodiments, the non-aqueous solution is miscible in the aqueous solution.

In some embodiments, the first and second amphiphilic block copolymers are mixed in a cold aqueous solution and the temperature of the mixture is then raised until polymeric micelles form.

In some embodiments, polymeric micelle nanoparticles are formed using solvent removal. The first and second amphiphilic block copolymers are combined in a non-aqueous solvent and the solvent is then removed. Removal of the solvent can be by evaporation, dialysis, filtration, of other methods of removing the solvent.

In some embodiments, polymeric micelle nanoparticles are formed using nanoprecipitation.

Following formation, the polymeric micelle nanoparticles can be purified. Purification can be used to remove solvent, remove non-micelle components, remove large aggregates, concentrate the polymeric micelle nanoparticles, or decrease polydispersity of the polymeric micelle nanoparticles. Purification methods include, but are not limited to, ultracentrifugation, density gradient centrifugation, ultrafiltration, sequential filtration, column purification, size exclusion chromatography, flow field-flow fractionation, hydrostatic filtration dialysis, immune affinity capture, precipitation, acoustic nanofiltration, and immuno-based microfluidic isolation.

### IV. Loading of Extracellular Vesicles

Extracellular vesicles (EVs) are membranous vesicles released by a variety of cells into the extracellular microenvironment. Based on the mode of biogenesis, these membranous vesicles can be classified into three broad classes (i), “exosomes” (exosomes are a specific subpopulation of membranous vesicles that excludes microvesicles and apoptotic bodies), (ii), microvesicles (also termed ectosomes), and (iii) apoptotic bodies. Exosomes are cell-derived vesicles originating from endosomal compartments produced during the vesicular transport from the endoplasmic reticulum to the Golgi apparatus. Extracellular vesicles are released extracellularly after the multivesicular bodies are fused with the plasma membrane. Extracellular vesicles are distinct from both ectosomes and apoptotic bodies in size, content, and mechanism of formation. Ectosomes are vesicles of various size (typically 0.1-1 mm in diameter) that bud directly from the plasma membrane and are shed to the extracellular space. Ectosomes have on their surface the phospholipid phosphatidylserine. Apoptotic bodies are formed during the process of apoptosis and are engulfed by phagocytes.

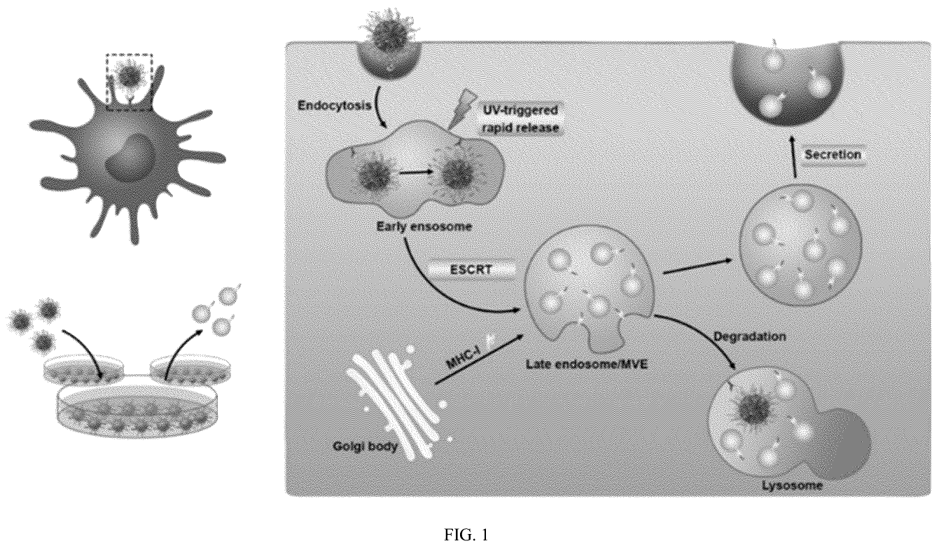

Described are methods of using the polymeric micelle nanoparticles to load one or more cargo molecules into extracellular vesicles (FIG. 1). The methods comprise contacting a cell with the polymeric micelle nanoparticle and incubating the polymeric micelle nanoparticle with the cell for a period of time sufficient to enable endocytosis of the polymeric micelle nanoparticle, exposing the cell to conditions suitable for cleavage of the reversible linkage, and collecting extracellular vesicles from the cell.

In some embodiments, contacting the cells with the polymeric micelle nanoparticles comprises adding the polymeric micelle nanoparticles into a culture medium containing the cells.

The polymeric micelle nanoparticles are incubated with the cell for a period of time sufficient for the polymeric micelle nanoparticles to be endocytoses and localize to early or late endosomes. In some embodiments, the polymeric micelle nanoparticles are incubated with the cell for a period of time sufficient for the polymeric micelle nanoparticles to be endocytosed and localize to early endosomes. In some embodiments, the polymeric micelle nanoparticles are incubated with the cell for a period of time sufficient for the polymeric micelle nanoparticles to be endocytosed and localize to late endosomes. The time needed for the polymeric micelle nanoparticles to endocytose and colocalized to early or late endosomes will vary depending on the cell type and the incubation conditions. The incubation time can be determined empirically for a given cell type and incubation condition. By way of example, the polymeric micelle nanoparticles can be labeled and analyzed for colocalization with known endocytic markers such as, but not limited to, EEA and Rab7. In some embodiments, the polymeric micelle nanoparticles are incubated with the cells under conditions suitable for endocytosis for about 10 minutes to about 4 hours, about 10 minutes to about 3 hours, about 10 minutes to about 2 hours, about 20 minutes to about 2 hours, about 30 minutes to about 2 hours, about 40 minutes to about 2 hours, about 50 minutes to about 2 hours, or about 1 hour to about 2 hours. In some embodiments, the polymeric micelle nanoparticles are incubated with the cells under conditions suitable for endocytosis for about 30 minutes.

Following internalization (endocytosis), the cargo molecule is released from the polymeric micelle nanoparticle. Release of the cargo molecule is facilitated by cleavage of the reversibly linker linking the cargo molecule to the polymeric micelle amphiphilic block copolymer. For pH sensitive reversible linkages, acidification of the endosome may release the cargo molecule. For light-sensitive reversible linkages, exposure of the cells to light results in release of the cargo molecule. For heat-sensitive reversible linkages, exposure of the cells to increased temperature results in release of the cargo molecule. In some embodiments, release of the cargo molecule is facilitated by adding an agent to the cells that facilitates or catalyzes cleavage of the reversible linkage.

In some embodiments, a light-sensitive reversible linker is used. The light-sensitive reversible linkage can be sensitive to near UV light. In such instances, exposure of the cells to near UV light results in release of the cargo molecule from the polymeric micelle nanoparticle.

In some embodiments, the cells (e.g., dendritic cells) are exposed to light having a wavelength of about 300 nm to about 700 nm. In some embodiments, the cells (e.g., dendritic cells) are exposed to near UV light having a wavelength of about 315 nm to about 500 nm. In some embodiments, the cells are exposed to near UV light having a wavelength of about 315 nm to about 490 nm, about 365 nm to about 488 nm, about 315 nm to about 405 nm, about 340 nm to about 390 nm, about 355 nm to about 375 nm, about 360 nm to about 370 nm, about 365 nm, about 405 nm or about 488 nm. In some embodiments, the cells are exposed to near UV light having a wavelength of about 365 nm.

In some embodiments, the cells (e.g., dendritic cells) are exposed to light having a wavelength of 300-700 nm at a light intensity of about 1 to about 10 W/cm², about 1 to about 5 W/cm², about 1.6 to about 4 W/cm², about 2 to about 3 W/cm², about 1.6 W/cm², about 2.4 W/cm², about 3.2 W/cm², or about 4.0 W/cm². In some embodiments, the cells are exposed to light having a wavelength of 300-700 nm at a light intensity of 2.4±0.6 W/cm², 2.4±0.5 W/cm², 2.4±0.4 W/cm², 2.4±0.3 W/cm², 2.4±0.2 W/cm², or 2.4±0.1 W/cm². In some embodiments, the cells (e.g., dendritic cells) are exposed to near UV light at 365 nm at a light intensity of about 1 to about 10 W/cm², about 1 to about 5 W/cm², about 1.6 to about 4 W/cm², about 2 to about 3 W/cm², about 1.6 W/cm², about 2.4 W/cm², about 3.2 W/cm², or about 4.0 W/cm². In some embodiments, the cells are exposed to near UV light at a light intensity of 2.4±0.6 W/cm², 2.4±0.5 W/cm², 2.4±0.4 W/cm², 2.4±0.3 W/cm², 2.4±0.2 W/cm², or 2.4±0.1 W/cm².

In some embodiments, the cells (e.g., dendritic cells) are exposed to light (e.g., 300-700 nm or near UV light) for an exposure duration of about 1 to about 30 minutes, about 5 to about 20, about 1, about 2, about 3, about 4, about 5, about 6, about 7, about 8, about 9, about 10, about 11, about 12, about 13, about 14, about 15, about 16, about 17, about 18, about 19, about 20 minutes. In some embodiments, the cells are exposed to light for an exposure duration of 10±5, 10±4, 10±3, 10±2, or 10±1 minutes. In some embodiments, the cells are exposed to light for an exposure duration of about 10 minutes

In some embodiments, the cells (e.g., dendritic cells) are exposed to 300-700 nm light at the above indicated intensities at a distance of about 1 to about 20 cm, about 2 to about 18 cm, about 5 to about 15 cm, about 8 to about 12 cm, about 9 to about 11 cm, about 5 cm, about 6 cm, about 7 cm, about 8 cm, about 9 cm, about 10 cm, about 11 cm, about 12 cm, about 13 cm, about 15 cm, or about 15 cm. In some embodiments, the cells are exposed to 300-700 nm light at a distance of 10±5 cm, 10±4 cm, 10±3 cm, 10±2 cm, or 10±1 cm. In some embodiments, the cells are exposed to 300-700 nm light at a distance of about 10 cm. In some embodiments, the cells (e.g., dendritic cells) are exposed to near UV light at the above indicated intensities at a distance of about 1 to about 20 cm, about 2 to about 18 cm, about 5 to about 15 cm, about 8 to about 12 cm, about 9 to about 11 cm, about 5 cm, about 6 cm, about 7 cm, about 8 cm, about 9 cm, about 10 cm, about 11 cm, about 12 cm, about 13 cm, about 15 cm, or about 15 cm. In some embodiments, the cells are exposed to near UV light at a distance of 10±5 cm, 10±4 cm, 10±3 cm, 10±2 cm, or 10±1 cm. In some embodiments, the cells are exposed to near UV light at a distance of about 10 cm.

Following release of the cargo molecule from the polymeric micelle nanoparticle in the early or late endosome, the cargo molecule can be incorporated into extracellular vesicles produced by the cell. The extracellular vesicles can then be harvested. In some embodiments, the extracellular vesicles are exosomes. A cargo molecule is loaded onto a EV if it is present in the interior of the EV, in the membrane of the EV (e.g., integral membrane protein), or associated with the surface of the EV.

The cell can be any eukaryotic cell from which extracellular vesicles can be derived. In some embodiments, the cell produces exosomes. The eukaryotic cell can be a mammalian cell. The mammalian cell can be an antigen presenting cell, a stem cell, an adult stem cell, an embryonic stem cell, a neural stem cell, an immune cell, a mesenchymal stem/stromal cell, an endothelial cell, or a fibroblast. The antigen presenting cell can be a dendritic cell, a macrophage, or B cell. The dendritic cell can be an immature dendritic cell or a mature dendritic cell. The dendritic cell can be a bone marrow derived dendritic cell. The dendritic cell can be a mammalian dendritic cell, a mouse dendritic cell, a rat dendritic cell, a rabbit dendritic cell, a pig dendritic cell, a sheep dendritic cell, a non-human primate dendritic cell, a human dendritic cell, a horse dendritic cell, a bovine dendritic cell, a dog dendritic cell, or a cat dendritic cell.

The described methods provide for on-demand release of cargo molecules into the endocytic pathway in a cell. Release of cargo molecules into the endocytic pathway provides for packaging of the cargo molecules into extracellular vesicles, such as exosomes. Subsequently, exosomes secreted by the cell contain the cargo molecules. The described methods can therefore be used to form extracellular vesicles carrying a cargo molecule. In some embodiments, the polymeric micelle nanoparticle reduces degradation of the cargo molecule and/or targeting molecule by the lysosome or lysosomal proteases.

The EVs produced by the methods described herein can be isolated using methods known in the art for isolating or purifying EVs. Such methods include, but are not limited to, sequential centrifugation (including ultracentrifugation), centrifugation using a sucrose density gradient or sucrose cushion, ultrafiltration, ExoQuick (System biosciences), Total Exosome Isolation Kit (Invitrogen), immunoaffinity capture, microfluidics-based isolation, and combinations thereof.

The described methods are compatible with conventional cell culture system for scaling up and GMP manufacturing. The described methods also provide for high-efficient and high-throughput production of therapeutic extracellular vesicles, including exosomes.

### V. Methods of Using Cargo-loaded Extracellular Vesicles

The ability to form extracellular vesicles having a desired cargo molecule (designer extracellular vesicles) can enhance the utility of EVs. Cargo-loaded EVs can be used for EV-based therapies and vaccines. The cargo-loaded EV can be used to delivery the cargo molecule to a cell or tissue in a subject. Cargo-loaded EVs can contain one or more targeting ligands that target the EVs to a tissue or cell type. In some embodiments, loading a cargo molecule, such as a cancer therapeutic (e.g., cancer immunotherapeutic), into an EV can reduce toxicity of a cargo molecule. In some embodiments, the EV is an exosome.

In some embodiments, the cargo-loaded EVs produced according to the described methods can be used to delivery a cargo molecule to a target cell.

In some embodiments, the cargo-loaded EVs produced according to the described methods can be used as therapeutics. The cargo-loaded EVs can be used in gene therapy, immunotherapy, cancer immunotherapy, precision medicine, or personalized medicine.

In some embodiments, the cargo-loaded EVs produced according to the described methods are able to modulate one or more functions of cells following contacting the cells with the EVs. In some embodiments, the cargo-loaded EVs produced according to the described methods are able to stimulate a response in a cell or tissue The cell can be in a subject. The cell can be a cell in a tissue or a tumor cell. In some embodiments, the cargo-loaded EVs produced according to the described methods are able to modulate immune function.

In some embodiments, the cargo-loaded EVs produced according to the described methods can be used to screen potential therapeutic compounds. The potential therapeutic compounds loaded into EVs according to the described methods. The cargo-loaded EVs are used to delivery the potential therapeutic compounds to cells in vitro or in vivo.

### VI. Kits

Described are compositions and kits comprising amphiphilic block copolymers suitable for using in forming the described polymeric micelle nanoparticles, the described polymeric micelle nanoparticles, or cargo-loaded EVs.

Any of amphiphilic block copolymers suitable for using in forming the described polymeric micelle nanoparticles, the described polymeric micelle nanoparticles, or cargo-loaded EVs may be packaged or included in a kit, container, pack, or dispenser. The amphiphilic block copolymers suitable for using in forming the described polymeric micelle nanoparticles, the described polymeric micelle nanoparticles, or cargo-loaded EVs sets may be packaged in pre-filled syringes, vials, or receptacles. The kit may also contain additional components for linking a cargo molecule or targeting molecule to an amphiphilic block copolymer.

A kit may further contain one or more of: instructions for use or a notice in a form prescribed by a governmental agency regulating the manufacture, use or sale of the products. The instructions may be associated with a package insert and/or the packaging of the kit or the components thereof.

### EXAMPLES

### Example 1. Synthesis of Amphiphilic Block Copolymers for Use Informing Polymeric Micelles Nanoparticles

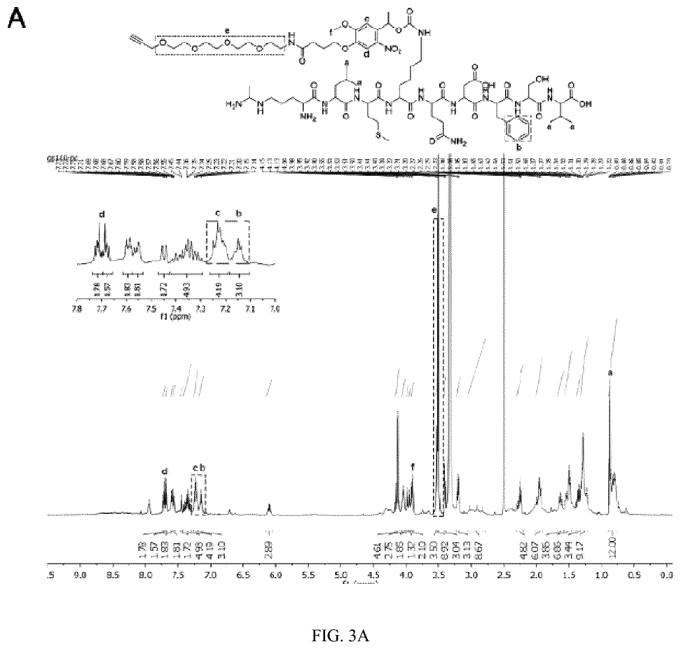

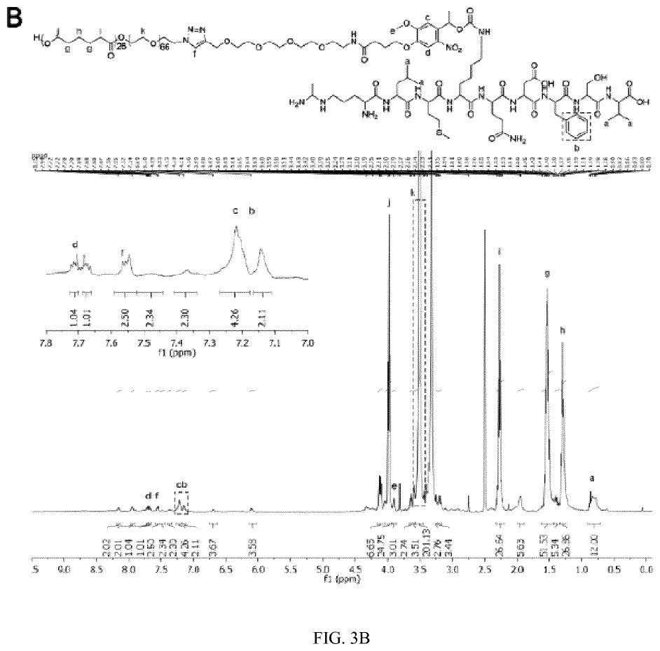

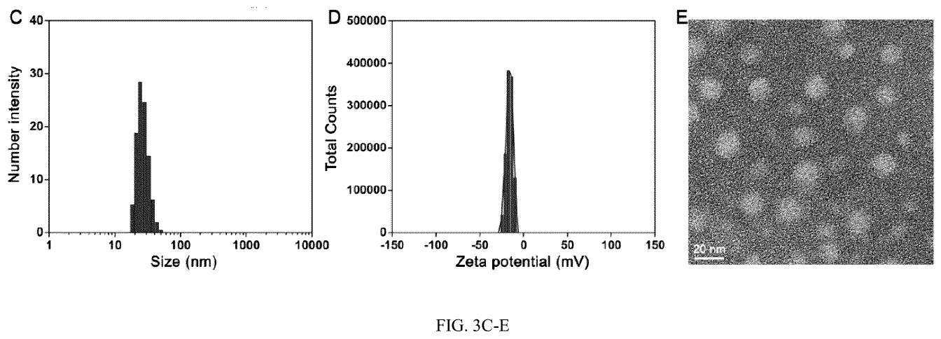

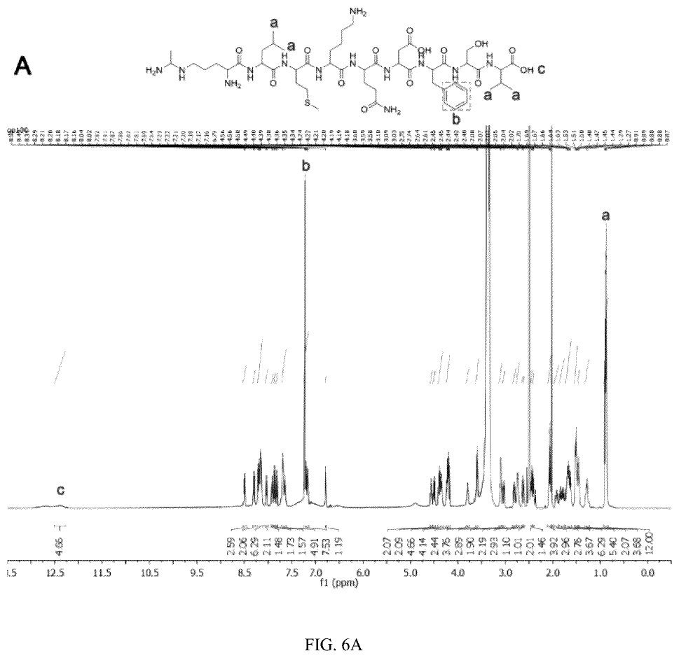

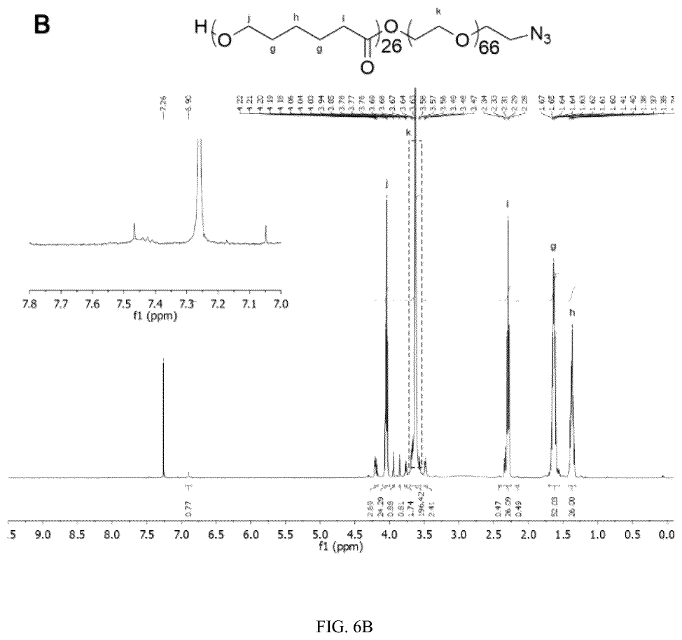

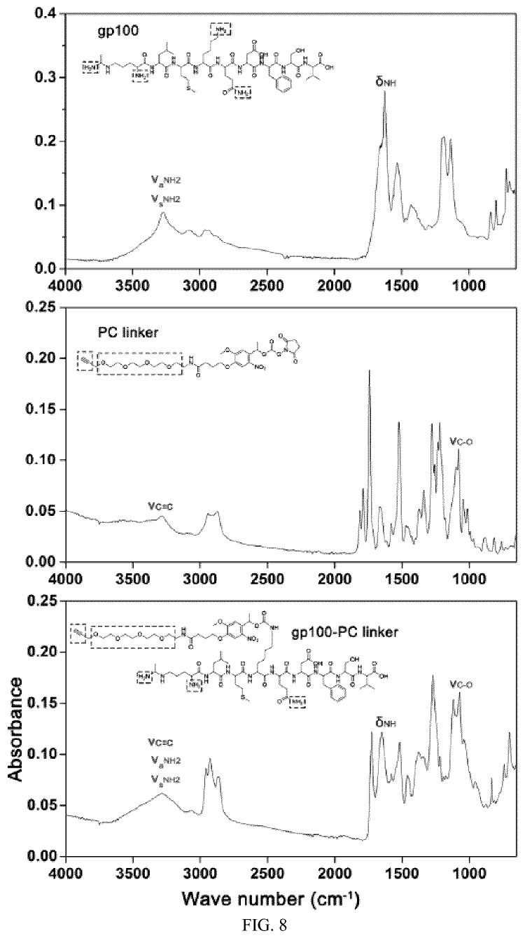

Synthesis of Alkynyl-Photocleavable (PC)-gp100: 4 mg of trifluoroacetate-stable gp100 (0.00356 mmol, 1.2 eq) was dissolved in anhydrous dimethylformamide (DMF, Sigma-Aldrich, USA) and photo-cleavable linker (2 mg, 0.00306 mmol, 1 eq) was introduced into the solution. Then, 0.62 μL of DIPEA (0.00356 mmol, 1 eq) was introduced into solution to remove the trifluoroacetate and expose the amine group on gp100. The solution was constantly stirred at room temperature for 8 h and protected from light to obtain alkynyl-PCgp100. The reaction was monitored by thin-layer chromatography and complete after 8 h reaction. For characterization, the product was purified by dialysis in deionized water in dialysis tubing (Molecular weight cutoff: 1000 Da) to remove DMF and unreacted reagents. The deionized water for dialysis was replaced every 8 h and the final solution was placed in freeze-dryer to evaporate the water to obtain alkynyl-PC-gp100. The alkynyl-PC-gp100 was identified using 400 Hz NMR and Fourier-transform infrared spectroscopy (FTIR). The NMR spectrum of the gp100 peptide is shown in FIG. 6A. The NMR spectrum of gp100-PC is shown in FIG. 3A. The FITR spectra of the gp100 peptide, PC linker, and gp100-PC-linker are shown in FIG. 8

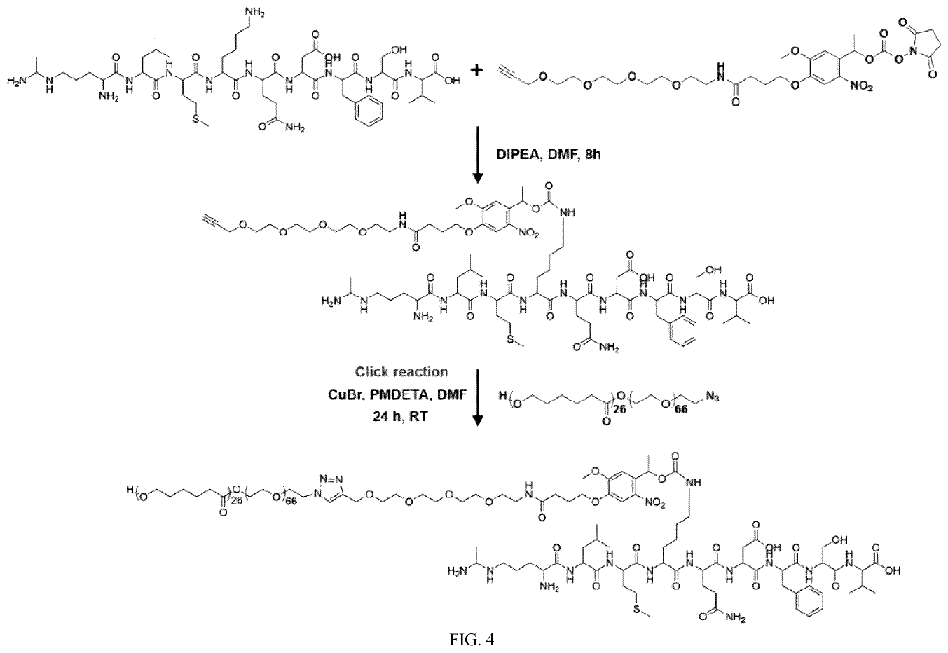

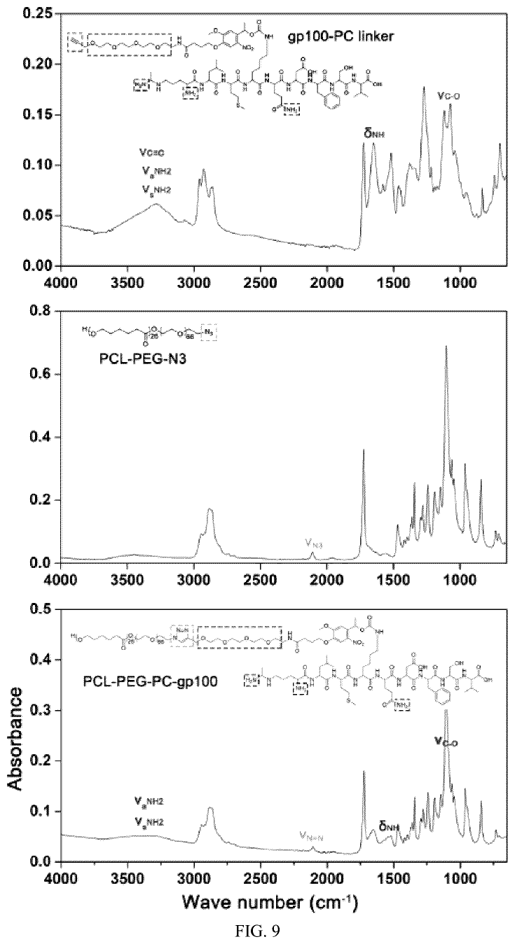

Synthesis of PCL-PEG-PC-gp100 (PPPG) via click chemistry. Synthesis of PPPG is illustrated in FIG. 4. The covalent conjugation between PCLPEG-N3 and alkynyl-PC-gp100 via click reaction was according to previously reported literature. [1] After synthesis of alkynyl-PC-gp100, PCL-PEG-N3 (18.36 mg, 0.00306 mmol, leq) dissolved in DMF was introduced into the alkynyl-PC-gp100 solution without purification, followed by addition of CuBr (0.44 mg, 0.00306 mmol, leq) pre-dissolved in DMF. To catalyze the click reaction, 0.63 μL of PMDETA (0.00306 mmol, 1 eq) was further added into the solution. The molar ratio of reagents of [PCL-PEGN3]:[alkynyl-PC-gp100]:[CuBr]:[PMDETA] was 1:1:1:1. The solution was stirred at room temperature for over 12 h and protected from light. After reaction (monitored by TLC), the product was purified by dialysis in deionized water in dialysis tubing (Molecular weight cutoff: 3500 Da). The deionized water for dialysis was replaced every 8 h and the final solution was placed in freeze-dryer to evaporate the water to obtain PCL-PEG-PC-gp100 powder. The PCL-PEG-PC-gp100 was identified using 400 Hz NMR and FTIR. The NMR spectrum of PCL-PEG-N3 is shown in FIG. 6B. The NMR spectrum of PPPG is shown in FIG. 3B. FTIR spectra of gp100-PC-linker, PCL-PEG-N3, and PCL-PEG-PC-gp100 are shown in FIG. 9.

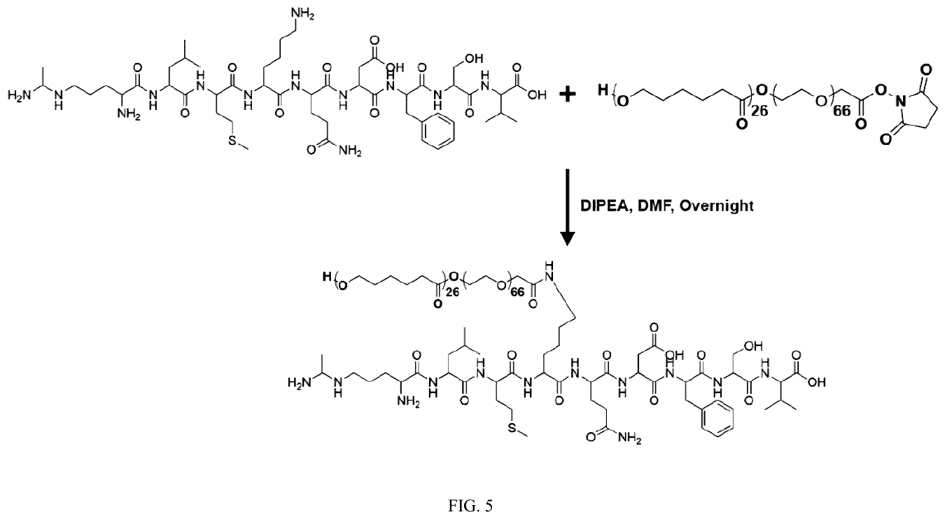

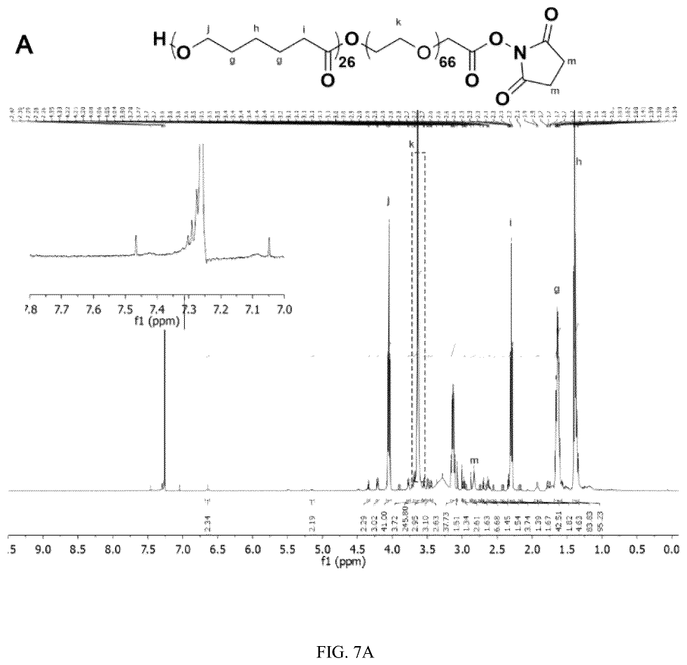

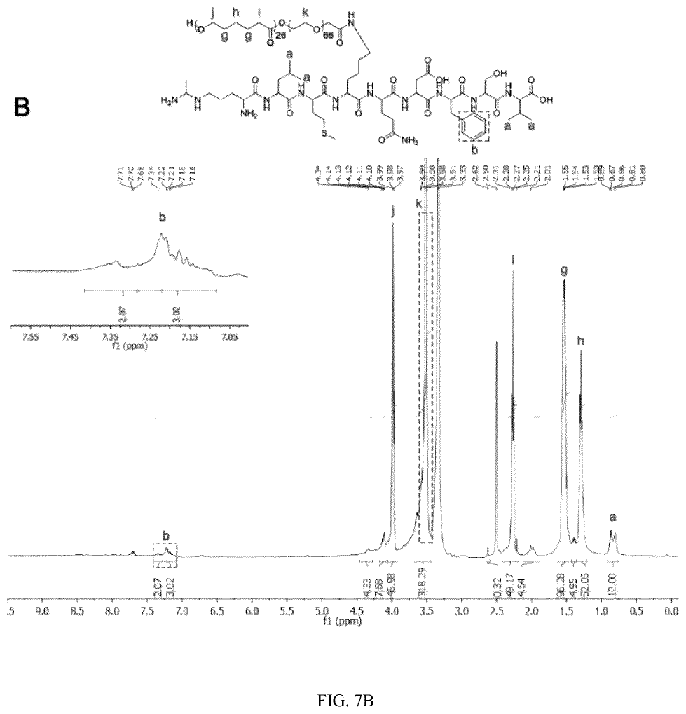

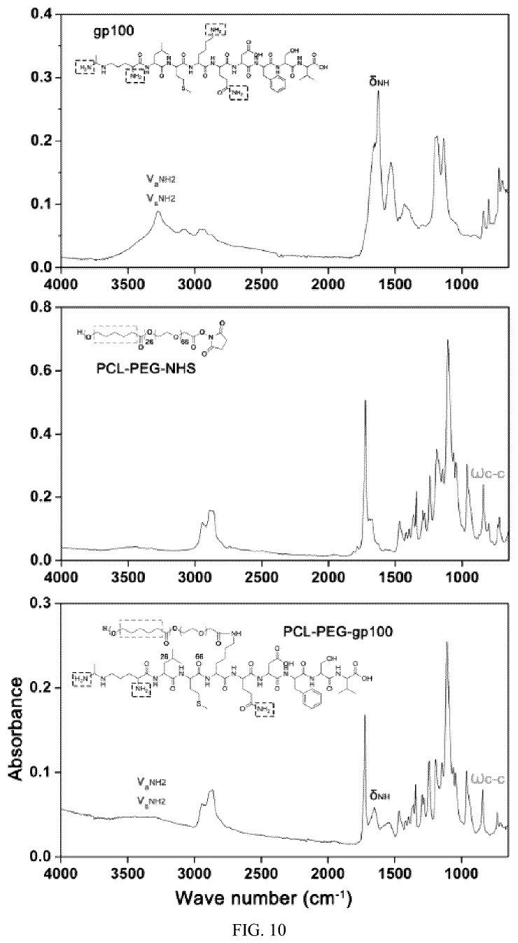

Synthesis of PCL-PEG-gp100 (PPG). Synthesis of PPG is illustrated in FIG. 5. 4 mg of trifluoroacetate-stable gp100 (0.00356 mmol, 1 eq) was dissolved in anhydrous DMF and added with 0.62 μL of DIPEA (0.00356 mmol, 1 eq) to remove the trifluoroacetate and exposure the amine group on gp100. Then, PCL-PEG-NHS (21.36 mg, 0.00356 mmol, 1 eq) dissolved in DMF was introduced into the solution. The mixed solution was constantly stirred at room temperature for 8 h and monitored completed by TLC. The product was purified by dialysis in deionized water in dialysis tubing (MWCO: 3500 Da) to remove DMF and unreacted reagents. The deionized water for dialysis was replaced every 8 h and the final solution was pre-frozen in −80° C. before drying in freeze-dryer to obtain PCL-PEG-gp100 powder. The NMR spectrum of PCL-PEG-NHS is shown in FIG. 7A. The NMR spectrum of PPG is shown in FIG. 7B. The FTIR spectra of gp100-PC-linker, PCL-PEG-NHS, and PCL-PEG-gp100 are shown in FIG. 10.

Synthesis of PCL-PEG-MAN (PPM): 1 mg of MAN (0.00367 mmol, 1.5 eq) was dissolved in anhydrous DMF and introduced with 21.2 mg of PCL-PEG-NHS (MW8000, 0.00265 mmol, 1 eq) pro-dissolved in DMF. The mixed solution was constantly stirred at room temperature for 8 h and monitored completed by TLC. The product was purified by dialysis in deionized water in dialysis tubing (Molecular weight cutoff: 3500 Da) to remove DMF and unreacted reagents. The deionized water for dialysis was replaced every 8 h and the final solution was pre-frozen in −80° C. before drying in freeze-dryer to obtain PCL-PEG-MAN powder.

### Example 2. Formation and Characterization of PPPG-PPM Polymeric Micelle Nanoparticles

Labeling of copolymer with fluorescein: 2.6 mg of PPPG (0.000339 mmol, 1 eq) was dissolved in DMF and added with 0.16 mg of FAM (0.000339 mmol, 1 eq) pre-dissolved in DMF. The mixed solution was constantly stirred at room temperature for 8 h and monitored completed by TLC. The product was purified by dialysis in deionized water in dialysis tubing (Molecular weight cutoff: 3500 Da). The deionized water for dialysis was replaced every 8 h and the final solution was pre-frozen in −80° C. before drying in freeze-dryer to obtain Cy5.5-labeled PPPG (PPPG-Cy5.5) powder. Similar procedures were performed to obtain Cy5.5-labeled PPG (PPG-Cy5.5) powder.

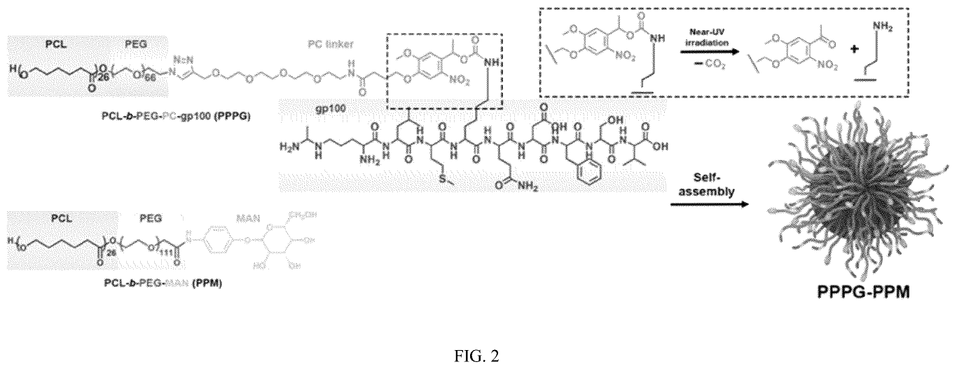

Synthesis of PPPG and PPM mixed micelles (PPPG-PPM): The synthesis of PPPG-PPM was through a nanoprecipitation method according to the previously reported literature. Briefly, PPPG (0.7 mg) and PPM (0.3 mg) were dissolved in 0.1 mL of dimethyl sulfoxide (DMSO), then the solution containing polymers was dropwise introduced into 2 mL of ultrapure water under vigorous stirring. After 10 min stirring, the solution was transferred into Amicon Ultra-4 centrifugal filter unit and centrifuged at 1500×g for 5 min to undergo ultrafiltration purification process. Filtered water from the outer tube was discarded and 2 mL of ultrapure water was added to the sample in the inside centrifugal filter unit. The filter unit was centrifuged again at 1500×g for 5 min. This process was repeated twice. After centrifugation, the remaining solution in the centrifugal filter unit was taken out and diluted to a final volume of 1 mL using ultrapure water to obtain PPPG-PPM mixed micelle solution. Formation of PPPG-PPM is illustrated in FIG. 2. Polymeric micelle nanoparticles using other amphiphilic block copolymers, cargo molecules, and targeting molecules can be formed using a similar process.

Synthesis of PPG-PPM, PPPG-PPC and PPG-PPC: To obtain PPG-PPM, PPG (0.7 mg) and PPM (0.3 mg) were dissolved in 0.1 mL of DMSO, then the solution containing polymers was dropwise introduced into 2 mL of ultrapure water under vigorous stirring. To obtain PPPG-PPC, PPPG (0.7 mg) and PCL-PEGCOOH (PPC, 0.3 mg) were dissolved in 0.1 mL of DMSO, then the solution containing polymers was dropwise introduced into 2 mL of ultrapure water under vigorous stirring. To obtain PPG-PPC, PPG (0.7 mg) and PPC (0.3 mg) were dissolved in 0.1 mL of DMSO, then the solution containing polymers was dropwise introduced into 2 mL of ultrapure water under vigorous stirring. Similar ultrafiltration purification process was performed to obtain PPG-PPM, PPPG-PPC and PPG-PPC mixed micelle solution.

Characterization of polymeric micelles: The hydrodynamic size and zeta potential of PPPG-PPM and control polymeric micelles were determined by dynamic laser scattering (ZS90 Zetasizer, Malvern Instruments). The morphology of these polymeric micelles was verified by transmission electron microscopy (TEM, FEI Spirit TEM 120 kV) imaging. Briefly, the ultra-thin copper grids coated with 400 mesh carbon film (FCF400-Cu-UB, Electron Microscopy Science, USA) was used with glow discharge treatment for 1 min before use. Then, 5 μL polymeric micelle samples were individually added onto glow-discharged grids and were quiescent for 2 mins at room temperature. The grids were washed with distilled water one time, then negatively stained with filtered 2% aqueous phosphotungstic acid for 2 mins and dried at room temperature before observation. The TEM imaging power was set at 120 kV by FEI Spirit G2 with a digital camera (Soft Image System, Morada and Gatan Orius SC 1000B CCD-camera).

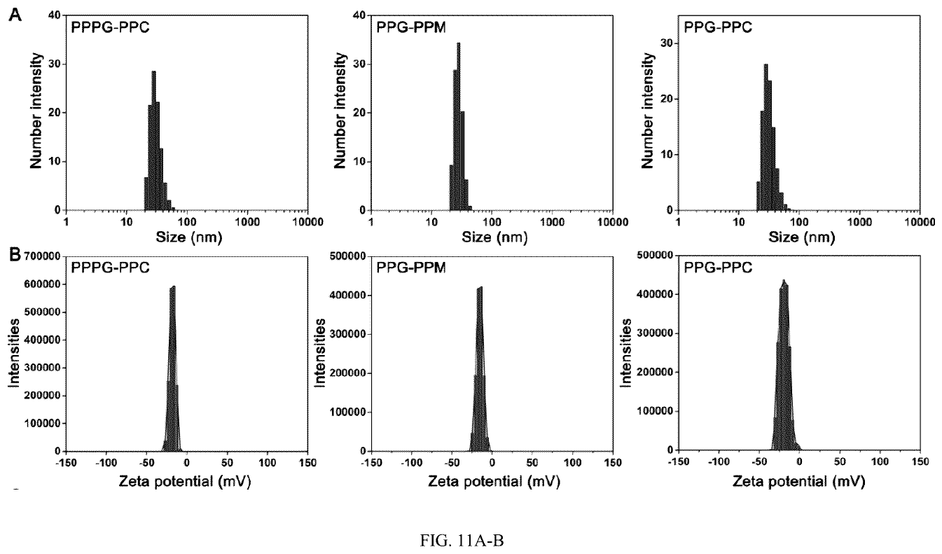

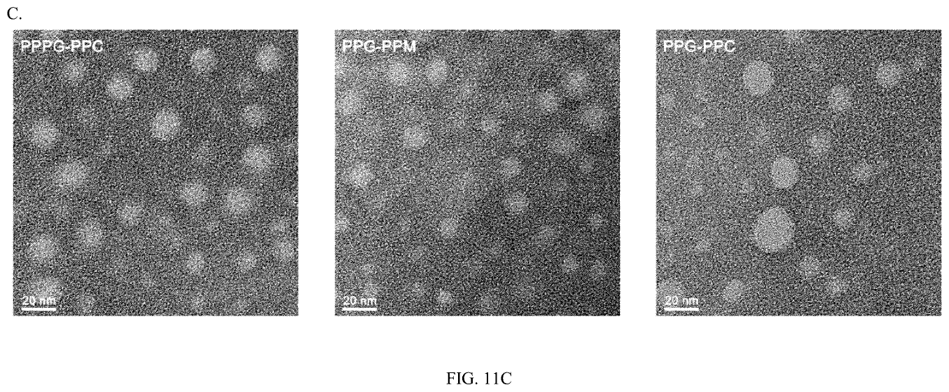

Number size distribution of PPPG-PPM polymeric micelle nanoparticles as determined by dynamic light scattering is shown in FIG. 3C. Zeta potential distribution of PPPG-PPM polymeric micelle nanoparticles as determined by dynamic light scattering is shown in FIG. 3D. A transmission electron micrograph of PPPG-PPM polymeric micelle nanoparticles after staining with 2% phosphotungstic acid is shown in FIG. 3E. Size distribution (A), zeta potential (B), and transmission electron micrographs (C) of control polymeric micelles is shown in FIG. 11, which illustrated the uniform and consistent polymer micelle nanoparticles.

### Example 3. Loading Dendritic Cells with Polymeric Micelle Nanoparticles

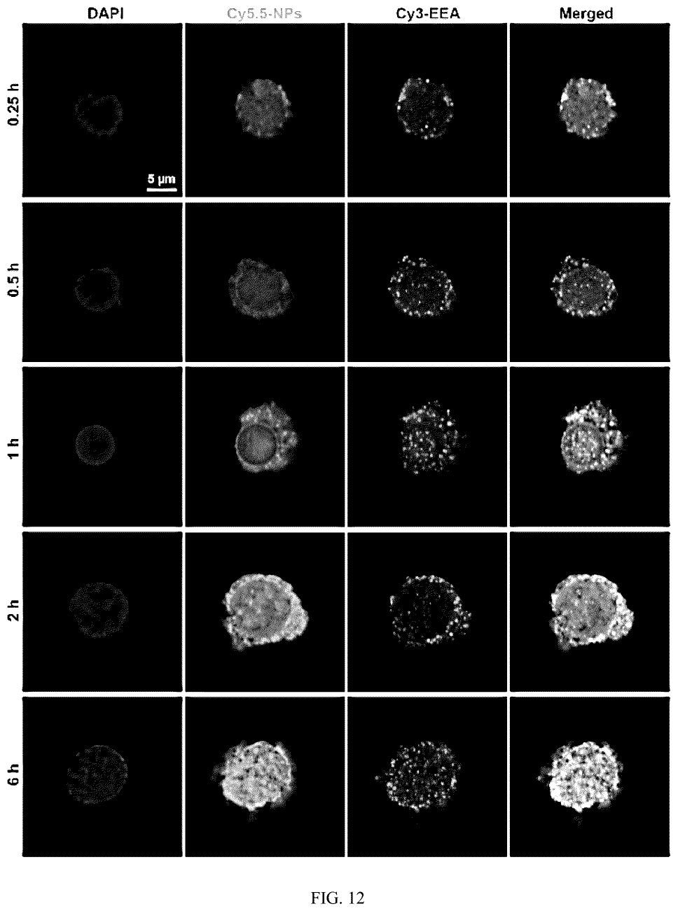

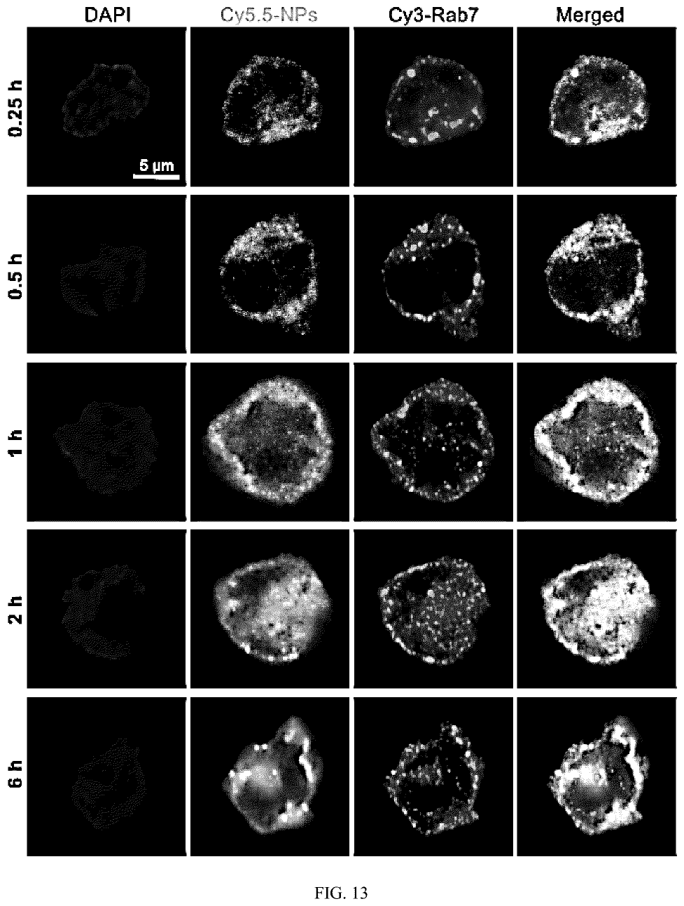

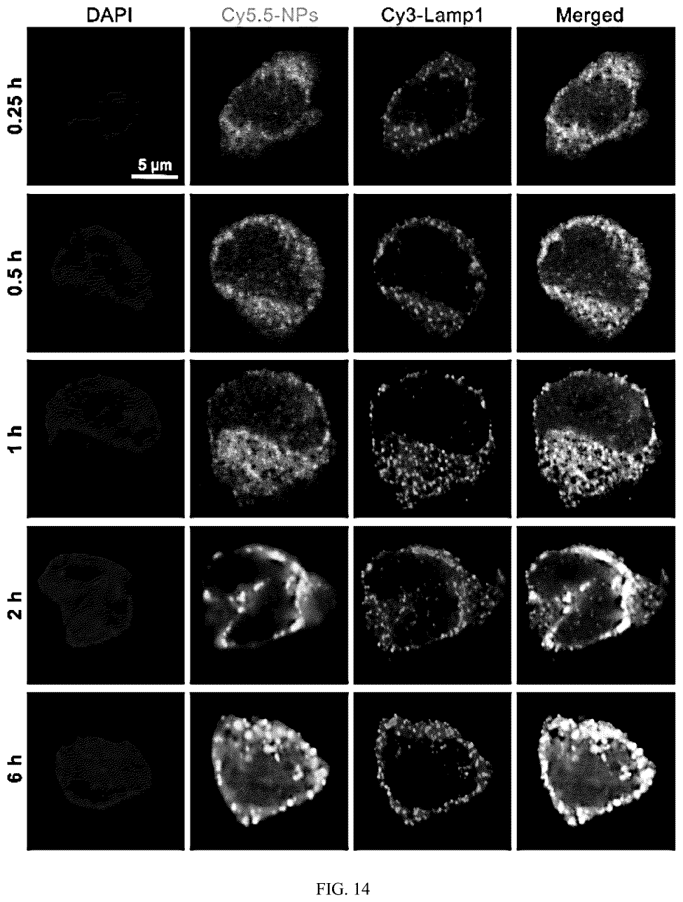

Uptake of the polymeric micelle nanoparticles by human mature dendritic cells was examined. Cy5.5-labeled polymeric micelle nanoparticles were prepared as described in Example 2. The labeled polymeric micelle nanoparticles were then incubated with the dendritic cells in media for various times. At various times, the dendritic cells are analyzed by microscopy. Immunofluorescence staining of polymeric micelle nanoparticles uptake and colocalization with early endosome (Cy3-labeled anti-EEA antibody), late endosome (Cy3-labeled anti-Rab7 antibody), and lysosome (Cy3-labeled anti-Lamp1 antibody) are shown in FIGS. 12, 13, and 14. The results indicate the polymeric micelle nanoparticles were endocytosed and co-localized with the early endosome by 1 hour. The results further indicate the polymeric micelle nanoparticles co-localized with the late endosome in 2 hours and the lysosome in 6 hours. This finding indicates that intracellular trafficking of the polymeric micelle nanoparticles to late endosomes may take place between 1-2 hours which is the process of sorting complex formation to pack into exosomes. Photo-controlled release of polymeric micelle nanoparticle carried gp-100 for specifical release into exosome packaging can thus be conducted in about one hour of incubation with dendritic cells. The derived exosomes will then carry enriched amount of gp-100 as engineered designer exosome therapeutics. Uptake by other cells is expected to occur similarly. The rate of uptake of the polymeric micelle nanoparticles by other cell types is readily determined. Once determined, the uptake rate can be used to determine when the stimulus to cleave the reversible linkage should be applied. to the cells. The data show that the described polymeric micelle nanoparticles can be used to load cells. Loading of the cells vis the endocytic pathway can then be used to add a desired cargo molecule into extracellular vesicles made by the cells, leading to engineered therapeutic-tailored exosomes.

### Example 4. Packaging into Polymeric Micelle Nanoparticles into Dendritic Cells and Generation of EVs

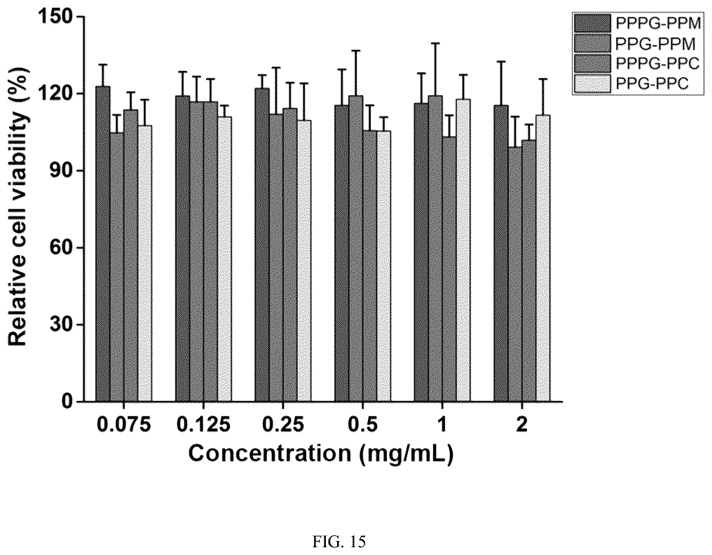

PPPG-PPM, PPG-PPM, PPPG-PPC, and PPG-PPC polymeric micelle nanoparticles were generated as described above and loaded into human dendritic cells (THP-1) at various concentrations. The DCs are then treated with light to cleave the photosensitive linkage in the polymeric micelle nanoparticles. DCs were then analyzed by MTT assay to determine cell viability. As shown in FIG. 15, greater than 90% viability was observed for each of the polymeric micelle nanoparticles at all of the concentrations tested, indicated little to no cytotoxicity.

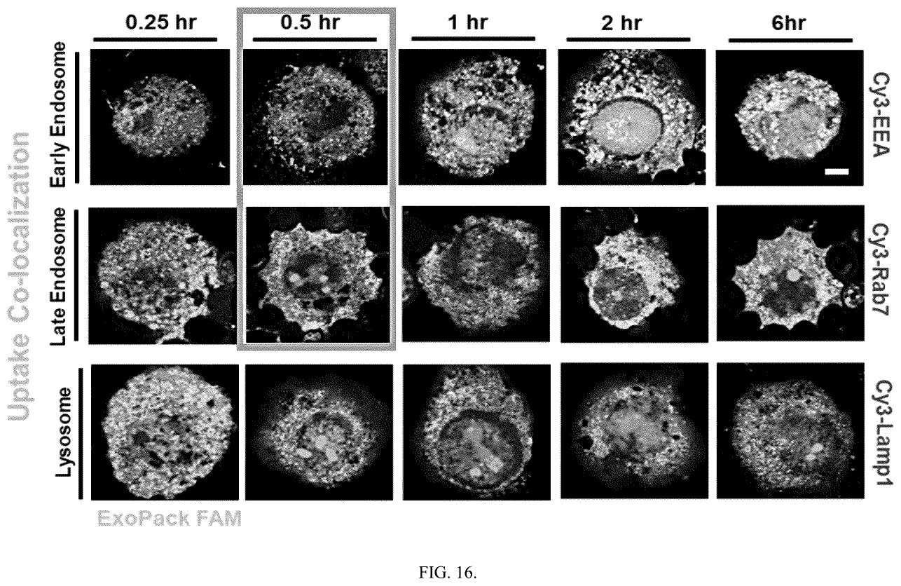

A timecourse analysis of cellular uptake of the polymeric micelle nanoparticles in shown in FIG. 16. Colocalization in early endosomes, later endosomes, and lysosomes was observed. The results indicate the majority of polymeric micelle nanoparticles were localized in early endosome after incubation with cells for 0.5 hr.

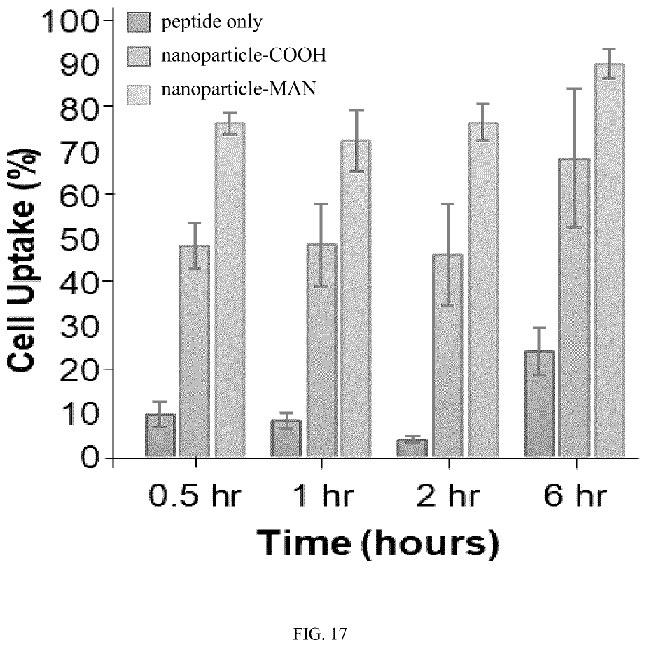

Cellular uptake of polymeric micelle nanoparticles modified with mannose ligand specific to dendritic cell surface receptor was compared with cellular uptake of polymeric micelle nanoparticles without surface modification. As shown in FIG. 17, polymeric micelle nanoparticles modified with mannose ligand were taken up be cells with a higher efficiency. Cells also internalized peptide in polymeric micelle nanoparticles modified with mannose ligand at a higher efficiency than peptide alone, indicating improved delivery of peptide to cells using targeting polymeric micelle nanoparticles.

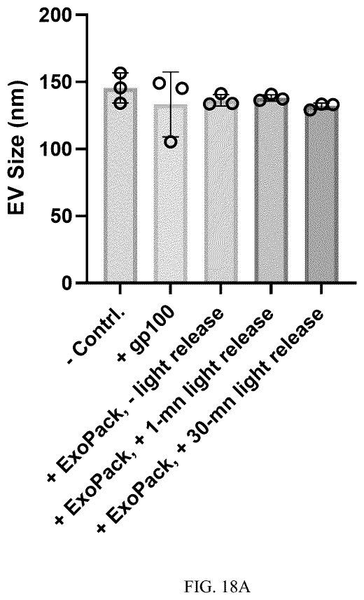

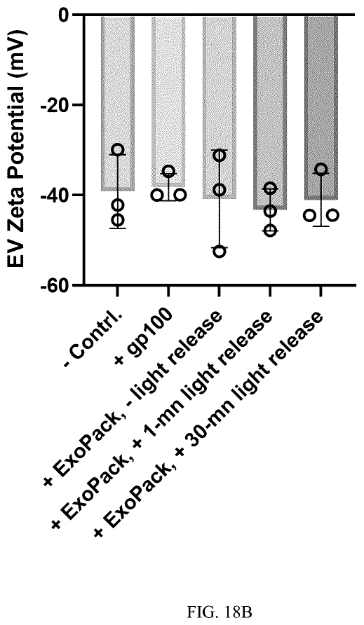

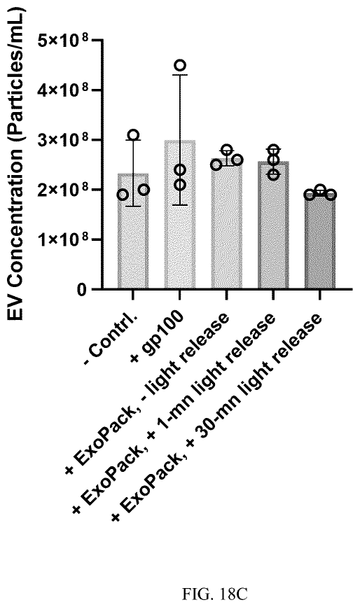

The size, zeta potential, and particle concentration in EVs harvested from dendritic cell following nanotransfection with polymeric micelle nanoparticles was analyzed. For comparison, native EVs from dendritic cells (i.e., without nanotransfection) and EVs from dendritic cells loaded with gp100 were also analyzed. For cells loaded with polymeric micelle nanoparticles (nanotransfection), cells were exposed to no light exposure, 1 minute light exposure, or 30 minute light exposure. As shown in FIGS. 18A-C, there was little to no difference in EV size, zeta potential, or number of EVs produces in any of the tested conditions. The results further show that loading of cells with polymeric micelle nanoparticles did not adversely affect EV production by the cells and the EVs produced with consistent with native EVs.

FIG. 18. The size (A), zeta potential (B), and particle concentration (C) characterization of harvested EVs after dendritic cell nanotransfection with Polymeric micelle nanoparticles, compared with native EVs from cells without transfection, and EVs from cellular uptake of gp-100. The light triggered on-demand cargo loading using Polymeric micelle nanotransfection was conducted under 1-min light exposure and 30-min light exposure, respectively. The results indicated that the size, zeta potential and secretion concentration of Polymeric micelle nanotransfected EVs are consistent with native EVs.

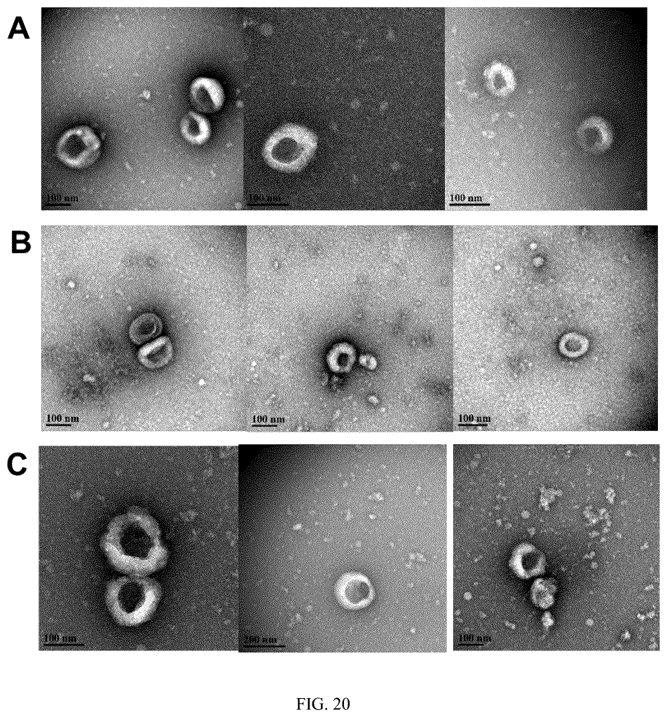

Morphology of harvested EVs was analyzed by transmission electron microscopy. As shown in FIG. 20, EVs harvested from dendritic cells following nantransfection with polymeric micelle nanoparticles and light-triggered cargo release (1 min or 30 minute light exposure) were consistent with native EVs.

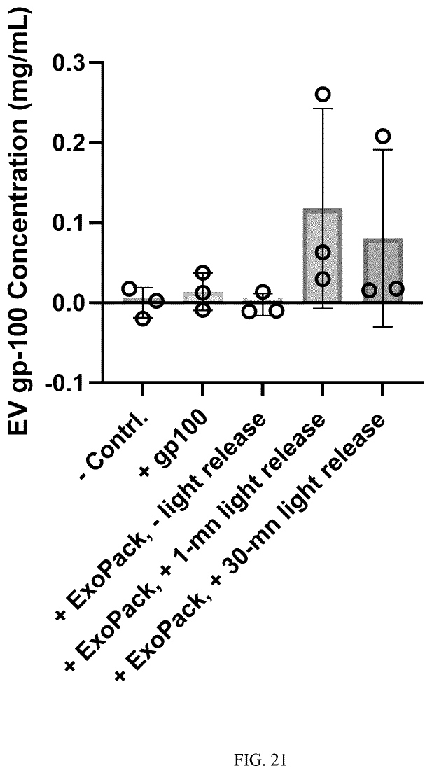

Dendritic cells were either untreated, incubate with gp100 peptide alone, or incubated with polymeric micelle nanoparticles containing gp100. Dendritic cells incubated with polymeric micelle nanoparticles containing gp100 were then exposed to light for 0 minutes (no release control), 1 minute or 30 minutes to release the gp100 from the nanoparticles. EVs were the collected and gp-100 concentration in the EVs was measured using ELISA kit from purified EV lysate. As shown in FIG. 21, EVs harvested from dendritic cells incubated with polymeric micelle nanoparticles containing gp100 were then exposed to light contained substantially more gp100.

1. A polymeric micelle nanoparticle comprising a first amphiphilic block copolymer having at least one reversible linkage to at least one cargo molecule and a second amphiphilic block copolymer covalently linked to a targeting molecule.

2. The polymeric micelle nanoparticle of claim 1, wherein the first amphiphilic block copolymer and the second amphiphilic block copolymer are diblock or triblock copolymers.

3. The polymeric micelle nanoparticle of claim 2, wherein the first amphiphilic block copolymer and the second amphiphilic block copolymer each comprise a hydrophobic block and a hydrophilic block.

4. The polymeric micelle nanoparticle of claim 3, wherein
(a) the hydrophobic blocks of the first and second block copolymers are the same; or
(b) the hydrophobic blocks of the first and second amphiphilic block copolymers are different.

5. (canceled)

6. The polymeric micelle nanoparticle of claim 3, wherein
(a) the hydrophobic blocks of the first and second amphiphilic block copolymers comprise polymers of the same monomer; or
(b) the hydrophobic blocks of the first and second amphiphilic block copolymers comprises polymers of different monomers.

7. (canceled)

8. The polymeric micelle nanoparticle of claim 3, wherein
(a) the hydrophilic blocks of the first and second amphiphilic block copolymers are the same; or
(b) the hydrophilic blocks of the first and second amphiphilic block copolymers are different.

9. (canceled)

10. The polymeric micelle nanoparticle of claim 3, wherein
(a) the hydrophilic blocks of the first and second amphiphilic block copolymers comprises polymers of the same monomer; or
(b) the hydrophilic blocks of the first and second amphiphilic block copolymers comprises polymers of different monomers.

11. (canceled)

12. The polymeric micelle nanoparticle of claim 3, wherein the at least one cargo molecule is linked to the hydrophilic block of the first amphiphilic block copolymer via the at least one reversible linkage.

13. The polymeric micelle nanoparticle of claim 3, wherein the at least one cargo molecule is linked to the hydrophobic block of the first amphiphilic block copolymer via the at least one reversible linkage, wherein the reversible linkage is cleaved in response to a drop in pH, an exposure to light, a reducing agent, a change in temperature, an enzyme, or an added agent.

14. (canceled)

15. The polymeric micelle nanoparticle of claim 13, wherein the reversible linkage comprises a photocleavable group comprising a nitrobenzyl group sensitive to light having a wavelength of 300-700 nm.

16. (canceled)

17. (canceled)

18. The polymeric micelle nanoparticle of claim 1, wherein the cargo molecule comprises a pharmaceutical molecule, a therapeutic compound, a drug, a Proteolysis targeting chimeric (PROTAC), a small molecule, a hormone, a cytokine, polypeptide, antibody, an antibody fragment, an antigen-binding polypeptide or molecule (e.g., scFv), or a nucleic acid molecule, or derivative thereof.

19. The polymeric micelle nanoparticle of claim 3, wherein the targeting molecule
(a) is linked to the hydrophilic block of the second amphiphilic block copolymer; and/or
(b) comprises a compound having affinity for a cell surface molecule, a cell receptor ligand, an antibody, an antibody fragment, an antigen-binding polypeptide, or the endosomal-sorting complex required for transport (ESCRT) machinery.

20. (canceled)

21. (canceled)

22. The polymeric micelle nanoparticle of claim 1, wherein the polymeric micelle nanoparticle is
(a) spherical in aqueous solution; and/or
(b) stable in an aqueous solution at about pH 7.4.

23. (canceled)

24. The polymeric micelle nanoparticle of claim 1, wherein the first amphiphilic block copolymer comprises PCLₙ₁-PEGₘ₁ wherein n1 is an integer from 2 to 250 and m1 is an integer from 2 to 750 and the second amphiphilic block copolymer comprises PCLₙ₂-PEGₘ₂ wherein n1 is an integer from 2 to 250 and m1 is an integer from 2 to 750.

25. The polymeric micelle nanoparticle of claim 24, wherein the first amphiphilic block copolymer comprises PCL₂₆-PEG₆₆ and the second amphiphilic block copolymer comprises PCL₂₆-PEG₁₁₁.

26. A method of forming extracellular vesicles containing a cargo molecule comprising:
(a) contacting a cell with the polymeric micelle nanoparticle of claim 1 and incubating the polymeric micelle nanoparticle with the cell for a period of time sufficient to enable endocytosis of the polymeric micelle;
(b) exposing the cell to conditions suitable for cleavage of the reversible linkage; and
(c) collecting extracellular vesicles from the cell.

27. The method of claim 26, wherein contacting a cell with the polymeric micelle nanoparticle and incubating the polymeric micelle nanoparticle with the cell for a period of time sufficient to enable endocytosis of the polymeric micelle, comprises loading the polymeric micelle nanoparticle into the cellular endosome pathway.

28. (canceled)

29. The method of claim 26, wherein incubating the polymeric micelle nanoparticle with the cell for a period of time sufficient to enable endocytosis of the polymeric micelle comprises incubating the polymeric micelle nanoparticle with the cell for about 10 minutes to about 2 hours.

30. (canceled)

31. (canceled)

32. (canceled)

33. An extracellular vesicle made by the method of claim 26.

34. The extracellular vesicle of claim 33, wherein the extracellular vesicle is
(a) used as a therapeutic; or
(b) used in drug delivery, gene therapy, immunotherapy, cancer immunotherapy, or precision medicine.

35. (canceled)

36. (canceled)
